(19) United States
(12) Patent Application Publication
(10) Pub. No.: US 2007/0184111 A1
(43) Pub. Date: Aug. 9, 2007

(54) Hybrid tablet

(75) Inventors: Scott Carl Harris, Brea, CA (US); Jones Y.C. Chan, Valencia, CA (US)

Correspondence Address: BLAKELY SOKOLOFF TAYLOR & ZAFMAN, 1279 OAKMEAD PARKWAY, SUNNYVALE, CA, 94085-4040, US

(73) Assignee: Pharmavite LLC

(21) Appl. No.: 11/348,102

(22) Filed: Feb. 3, 2006

(51) Int. Cl. A61K 9/24

(52) U.S. Cl. 424/471

(57) ABSTRACT

A hybrid tablet and method are provided. The tablet may have an inner core and an outer layer. The outer layer includes a first agent, the outer layer having a first dissolution rate. The inner core includes a second agent, the inner core having a second dissolution rate different from the first dissolution rate. The method includes forming the core having a second agent and forming the outer layer having a first agent. The core is formulated for timed-release of the second agent. The outer layer is formulated to elicit a salivary response in a mouth. The hybrid tablet is formed by applying the outer layer to the core.

### BACKGROUND

1. Field

A hybrid tablet.

2. Background

Delivery of medicinal, nutritional and other similar agents for absorption within a person's body may be accomplished in any number of ways. For example, the agent may be a component of an oral, topical or injectable delivery system. Of the available delivery systems, oral delivery, such as by way of a tablet, is often the preferred system. Such a delivery system offers the least invasive and possibly least expensive of the available systems. Nevertheless, such a delivery system is not without its faults. In the case of a conventional tablet, the agent is typically distributed uniformly throughout the tablet. Thus, where the agent has an unpleasant flavor, this flavor is inevitably experienced by the person once the tablet is placed upon a user's tongue. Moreover, where the agent has an aftertaste, even after the tablet is swallowed, the taste of the agent lingers in a person's mouth.

In addition, a person is often required to take multiple tablets in treating a single condition. This may occur where multiple site absorption of the agents is desired. Typically, each tablet is formulated such that it dissolves or disintegrates at a particular rate (i.e. dissolution rate) within the body. Upon dissolution of the tablet, agents within the tablet are released (i.e. release rate) at sites (e.g. buccal cavity, intestines, etc.) within the body. Once the agents are released, they may be absorbed at the site where they are released. Thus, where the tablet is a uniform tablet having a single dissolution rate, agents within the table are released at the same rate. Thus, even though one agent may be more effective when absorbed within the buccal cavity and another more effective when absorbed within the intestine, both agents are released at the same site thereby reducing the effectiveness of the agent.

Separate tablets may further be necessary where the tablet is to large to swallow when all the agents are included within the tablet or where the agents are highly reactive and react with other agents found in the tablet. Since agents are generally blended together to form the tablet, those which are reactive may react with one another prior to being delivered to the body. Such a result may reduce the effectiveness of the agents within the body's system.

Moreover, conventional tablets are often inconvenient since a liquid must be provided to swallow the tablet. Often times, the user may not have a liquid available. In such an instance, the user must either wait to take the tablet until they find a liquid or attempt to swallow the tablet without the aid of a liquid. Although tablets in a chewable form, such as for example Flintstone's vitamins, may overcome this aspect, a chewable tablet form is not desirable where agents within the core have an unpleasant flavor. Thus, either option may frustrate or cause discomfort to the user.

A tablet that allows for delivery of a number of agents without the need for a liquid to assist in swallowing the tablet remains desirable.

### BRIEF DESCRIPTION OF THE DRAWINGS

The following illustration is by way of example and not by way of limitation in the figures of the accompanying drawings in which like references indicate like elements. It should be noted that references to “an” or “one” embodiment in this disclosure are not necessarily to the same embodiment, and such references mean at least one.

FIG. 1 illustrates one embodiment of a hybrid tablet.

FIG. 2 illustrates one embodiment of a hybrid tablet including beads in an outer layer.

FIG. 3 is a flow chart of one embodiment of a method for forming a hybrid tablet.

### DETAILED DESCRIPTION

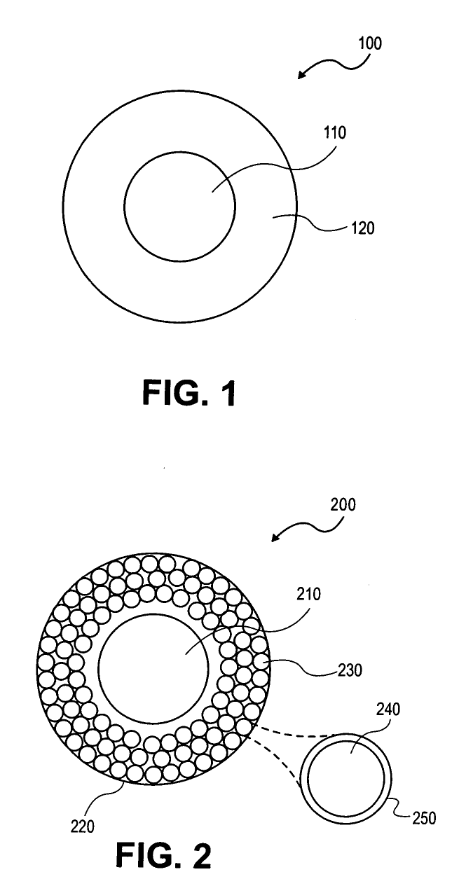

A tablet is disclosed. In one aspect, the tablet is directed at controlling the release of an agent within the body. FIG. 1 illustrates one embodiment of a hybrid tablet. A circular-shaped tablet is shown. Other shapes (e.g., oval, rectangular etc.) may also be suitable. Tablet 100 includes outer layer 120 and inner core 110. Outer layer 120 is formed around inner core 110. Outer layer 120 may have a first dissolution rate. Inner core 110 may have a second dissolution rate. The first dissolution rate may be greater (i.e. faster release) than the second dissolution rate. In an alternative embodiment, the first and second dissolution rates may be substantially the same. Still further, the second dissolution rate may be greater than the first dissolution rate. In one embodiment, tablet 100 may be administered once a day. In an alternative embodiment, tablet 100 may be administered any number of times a day found to be acceptable to the diet.

It is believed the separation of outer layer 120 and inner core 110 may reduce the number of tablets required per dose. In one aspect, the separate layers having different dissolution rates may accommodate multiple site absorption (i.e., sublingual, gastric and/or intestinal) of several agents within a single tablet. Still further, in conventional uniform tablets, reactive agents are often administered in separate tablets to minimize the possibility of the agents reacting with one another prior to delivery to the body. In a similar manner separation of outer layer 120 and inner core 110 can inhibit interaction of such reactive agents since the agents may be separated into one of the outer layer and inner core. In this aspect, the need for multiple tablets is eliminated.

Outer layer 120 may be made of a technology known in the art to dissolve rapidly (i.e. melt away) within the mouth. Suitable technologies, may include, but are not limited to, for example, ZYDIS®, OraSolve, ADVATAB®, FLASHDOSE®, WOWTAB®, PHARMABURST® and Lyco. These technologies may be incorporated into available drug products to provide a quick dissolving delivery platform for the agent. For example, PHARMABURST® is a co-processed excipient system designed to be used with a blend of agents and compressed along with the agent into a tablet form. A representative dissolution rate of an outer layer of a hybrid tablet incorporating a melt-away technology is a dissolution rate (a first dissolution rate) of approximately two minutes or less. In another embodiment, the first dissolution rate may be between approximately five seconds and approximately two minutes. In a further embodiment, the first dissolution rate may be between approximately three seconds and approximately 30 seconds. Still further, the first dissolution rate may be between approximately 15 seconds and approximately 20 seconds.

In one embodiment, outer layer 120 may include a first agent released upon dissolution of the outer layer at the first dissolution rate. Outer layer 120 may include any number of first agents. The first agent may be, but is not limited to, for example, a vitamin, a therapeutic, a mineral, an herbal extract, an amino acid, a stimulant and/or a drug. Examples of vitamins include, but are not limited to, ascorbic acid, Coenzyme Q10 (Co Q10), vitamin A, vitamin B, vitamin D and vitamin K. Examples of therapeutics may include, but are not limited to, a fish oil and a glucosamine. Examples of minerals may include, but are not limited to, a zinc gluconate, a ferrous sulfate, a chromium picolinate and a magnesium oxide. Examples of herbal extracts may include, but are not limited to, Yerba Santa extract and cinnamon extract. An example of an amino acid is, but is not limited to, an L-arginine. An example of a stimulant may include, but is not limited to, caffeine. Examples of a drug may include, but are not limited to, a nitroglycerin and dextromethorphan. In an embodiment where inner core 110 includes a probiotic, outer layer 120 may serve as a barrier layer helping to maintain organism counts within the probiotic.

In one embodiment, the first agent may be included in outer layer 120 in an amount sufficient to achieve the desired effect of the agent when absorbed within the body of the user. In one embodiment, outer layer 120 may include a first agent in a dietary acceptable amount. For example, in an embodiment where the tablet is intended to be administered once per day, and the outer layer includes a first agent that is the vitamin ascorbic acid, the outer layer may include approximately 83.48 weight percent (wt. %) Pharmaburst-SPI, 7.37 wt. % ascorbic acid, 4.59 wt. % citric acid anhydrous, 0.29 wt. % sucralose, 3.10 wt. % cherry flavor (VIRGINIA DARE®) and 1.13 wt. % sodium stearyl fumerate and/or 1.13 wt. % magnesium stearate. The amount of ascorbic acid (vitamin C) in the outer layer represents 100 percent of the recommended daily allowance (RDA) of the vitamin or 60 mg.

In one aspect, outer layer 120 may have a property rendering it palatably acceptable for dissolution in a buccal cavity. For example, an outer layer including 400 IU of vitamin D as a first agent and 60 mg of ascorbic acid is considered sufficient to render the outer layer palatably acceptable for dissolution in the buccal cavity. In one embodiment, outer layer 120 may further include, for example, a flavoring agent and/or sweetener such as sucralose. The flavoring agent may include, but is not limited to, an organic acid, a cherry flavor such as VIRGINIA DARE®, a grape flavor and/or other similar flavoring agents.

Although in healthy individuals the mouth naturally produces saliva to aid in swallowing and digestion, this amount of saliva is typically not sufficient to allow for comfortable swallowing of a tablet or capsule in the absence of a liquid. In this aspect, outer layer 120 may include an effective amount of an organic acid suitable for eliciting a salivary response within a mouth to aid in swallowing the inner core in the absence of liquid. An organic acid is characterized in this regard by a carboxyl (—COOH) group. Examples include, but are not limited to, citric acid, malic acid, adipic acid, fumaric acid, ascorbic acid and tartaric acid. For example, an effective amount of an organic acid may be approximately 60 mg of ascorbic acid to elicit a salivary response for an outer layer having 400 IU of vitamin D as a first agent. It is believed that such a proportion of organic acids stimulate a maximum salivary response within the buccal cavity while still being palatably acceptable to the user. Thus, the user may retain the tablet within the buccal cavity for a period of time and then swallow the tablet without the aid of a liquid.

FIG. 2 illustrates another embodiment of a hybrid tablet. Tablet 200 includes outer layer 220 and inner core 210. Outer layer 220 may include compacted beads 230. In other words, a tablet having an outer layer including enrobing beads 230 may have a first dissolution rate in the sense that beads 230 go from a collected or compacted state (e.g., to form a solid tablet) to a stipulated state and a separate dissolution rate in the sense of the individual beads dissolving to release the agent. Beads 230, may be, for example, sustained or timed release beads similar to those used in the common cold medication known as CONTAC®. A magnified view of bead 230 is further shown in FIG. 2 to illustrate such a bead. Bead 230 is shown having an outer coating 250 which enrobes an agent 240. In this aspect, outer coating 250 coat an agent, for release upon dissolution of the beads at the dissolution rate. Agent 240 may include, but is not limited to, for example, the first agents described above with reference to outer layer 120 of FIG. 1. Alternative agents that may be utilized in the form of enrobed beads in outer layer 220 of tablet 200 include, but are not limited to, niacin (vitamin B) and dextromethorphan. The dissolution rate of a bead may be substantially the same as or different than the first dissolution rate.

The palatability and dissolution rate of outer layer 120 (FIG. 1) or outer layer 220 (FIG. 2) allows several agents to be included in a tablet while still providing a tablet size that can be comfortably swallowed. For example, the inner core may include any number of agents, bringing the size of the inner core up to a maximum tablet size suitable for swallowing by the user. In one embodiment, the addition of the outer layer can bring the tablet size above that which could be comfortably swallowed. This does not present a problem in the instant tablet, however, since the outer layer may be dissolved within the buccal cavity and then the inner core swallowed. In this regard, the tablet is able to combine a large number of agents or a large amount of one or more agents (large in the sense that the volume of the tablet is larger than a typical tablet having multiple agents intended for direct swallowing) while still maintaining a suitable swallow size.

Referring to the embodiment of FIG. 1 and/or the embodiment of FIG. 2, inner core 110 or inner core 210 may include an agent (a second agent). Inner core 110/210 may include any number of second agents. The second agent may be released upon dissolution of the inner core at a dissolution rate (the second dissolution rate). The second dissolution rate may be such that the rate of release of the second agent is immediate and/or sustained. Still further, the second dissolution rate may be such that the second agent is released over a period of approximately 24 hours or less. In another aspect, the second dissolution rate may be such that the second agent is released over a period of between approximately two minutes and six hours. In another aspect, the second dissolution rate may be such that the second agent may be released over a period of between approximately two minutes and 24 hours.

The second agent may be in a liquid and/or solid form. The second agent may be, but is not limited to, one or more of a mineral, a vitamin, a probiotic and/or an herbal extract. Suitable minerals include, but are not limited to, calcium (e.g., a calcium carbonate and/or calcium citrate), zinc, selenium and iron. In one embodiment, the second agent may be a mineral trituration. The mineral trituration may include, but is not limited to, for example manganese, silicon, calcium, phosphorus, chromium, molybdenum, selenium, vanadium, nickel, boron, copper, iron and/or calcium. Suitable vitamins include, but are not limited to, ascorbic acid, CoQ10, vitamin A, vitamin B, vitamin D and/or vitamin K. A suitable probiotic includes, but is not limited to, acidophilus. Suitable herbal extracts may be, but are not limited to, valarian, green tea extract, cinnamon extract, Echinacea extract, ginseng extract and/or Andrographis extract.

In an embodiment where a tablet includes an outer layer and inner core where the outer layer is intended to be dissolved (e.g., completely dissolved) in the buccal cavity, it may be desirable to inhibit the dissolution of the inner core in the buccal cavity. In such case, the inner core may include a delay release agent. In one embodiment, the delay release agent may be a stearic acid. Stearic acid has hydrophobic properties which inhibit liquid in the stomach or intestine from dissolving the second agent within the core. In one embodiment, an amount of stearic acid and the second agent may be mixed together and included within the core. In one embodiment, the second agent may be coated with stearic acid and included within the core.

In one embodiment, the second agent may be included in the inner core in an amount sufficient to achieve the desired effect of the agent when absorbed within the user's body. In one embodiment, the inner core may include second agents in a dietary acceptable amount. In an embodiment where the tablet is administered once a day, a sufficient amount of the second agents may be approximately 1 mg manganese, 505 mcg silicon, 32.4 mg calcium, 2.2 mg phosphorous, 75 micrograms chromium, 37.5 mcg molybdenum, 10 mcg selenium, 12.5 mcg nickel, 75 mcg boron, 1 mg copper, 18 mg iron and/or 5 mcg vanadium.

In one embodiment, the dissolution rate of the inner core may have an immediate or sustained dissolution rate. The inner core may be a bilayer tablet or a single layer tablet. Where the inner core is a bilayer tablet, the bilayer may have a first layer and a second layer. In one embodiment, the first layer surrounds the second layer. In this aspect, the first layer may have a dissolution rate and the second layer may have a dissolution rate. The dissolution rate of the second layer may be different from the dissolution rate of the first layer. In an alternative embodiment, the dissolution rate of the second layer may be substantially the same as the dissolution rate of the first layer. In one embodiment, the dissolution rate and/or the dissolution rate of the first layer of the bilayer inner core may be substantially the same as the dissolution rate of the outer layer of the tablet.

In an embodiment where the inner core is a bilayer, one of the above-described second agents may be included in the first layer and another may be included in the second layer. Still further, any number of the second agents may be included in the first layer and the second layer. In this aspect, agents may be released from the core at the second dissolution rate or a third dissolution rate to facilitate multiple sight absorption.

In one embodiment, inner core 110 is a soft gel capsule or gum. A soft gel capsule or gum can permit a higher concentration of the second agent to be delivered than where the inner core is a solid. In this aspect, the second agent may be selected from the group of second agents discussed above. The second agent may be in a liquid form. In an alternative embodiment, the second agent may be, but is not limited to, for example, a vitamin, a therapeutic, a lecithin, an herbal extract and/or a carotenoid. The vitamin may be, but is not limited to, for example, a beta-carotene (vitamin A), and/or vitamin E. The therapeutic may be, but is not limited to, for example, a fish oil (DHA/EPA). The herbal extract may be, but is not limited to, for example, evening primrose oil.

It is believed the multiple dissolution rates of the described tablet allow for multiple site absorption of agents. For example, the tablet may include an outer layer having a first dissolution rate including beads having a separate dissolution rate and the core may be a bilayer, each layer having different dissolution rates (e.g., second and third dissolution rates, respectively). The first dissolution rate (e.g. five seconds) may be greater (i.e. faster release) than a dissolution rate of the beads (e.g. 15 seconds), the beads may have a dissolution rate greater than the third dissolution rate (e.g. two minutes), and the third may be greater than the second dissolution rate (e.g. 24 hours). Thus, it is possible for agents within each of the layers to be released at four different rates. In this aspect, agents may be introduced into each layer depending upon where within the body release is desired. For example, agents to be released within the buccal cavity may be placed in the outer layer and/or beads of the outer layer. Agents to be released within the gastrointestinal tract may be placed in one of the bilayers of the core. In this aspect, when the tablet is placed in the persons' mouth, agents in the outer layer are released in the buccal cavity within five seconds and agents in the beads are released within 15 seconds of placing the tablet in the users mouth while those agents in the bilayer are retained within the tablet. As the tablet is swallowed and travels through the lower organs such as a stomach or intestine, the remaining agents are released from the bilayer.

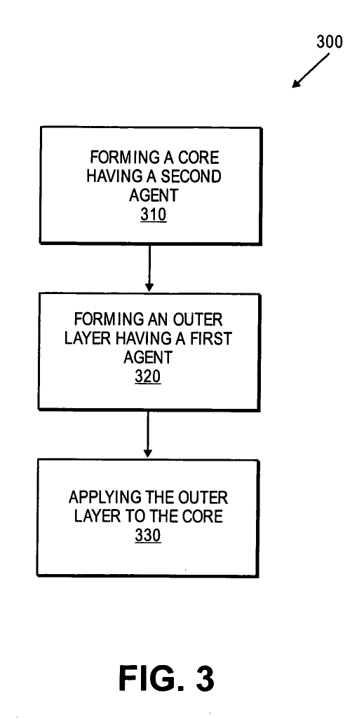

A method is provided for forming the hybrid tablet 300. FIG. 3 illustrates this method. In one embodiment, a core is formed having a second agent (block 310), wherein the core is formulated for timed-release of the first agent. An outer layer is formed having a first agent (block 320), the outer layer elicits a salivary response when placed in a user's mouth. The outer layer is applied to the core (block 330) to form a hybrid tablet.

The following specific examples are set forth to illustrate a method for forming the core and outer layer.

### EXAMPLE I—OUTER LAYER

| | Amount (871 gram tablet |
|---|---|
| Ingredient | weight) |
| Vitamin D3, 100,000 IU/gram | 1.82% |
| PHARMABURST ® - SPI | 83.48% |
| Ascorbic Acid, 95% | 7.37% |
| Citric Acid Anhydrous | 4.59% |
| Sucralose | 0.29% |
| Cherry Flavor, VIRGINIA | 3.10% |
| DARE ® | |
| Sodium Stearyl Fumerate or | 1.13% |
| Magnesium Stearate | |

In one embodiment shown in Example I, effective amounts of second agents and excipients of the outer layer are illustrated.

### EXAMPLE II—INNER CORE

| | Amount (180 gram tablet | |
|---|---|---|
| Ingredient | weight) | |
| Manganese sulfide powder | 1.82% | |
| Manganese | 1 | mg |
| Silicon dioxide | 0.63% | |
| Silicon | 500 | mcg |
| Magnesium stearate | 1.44% | |
| Mineral trituration | 5.91% | |
| Calcium | 2.4 | mg |
| Phosphorous | 2.2 | mg |
| Chromium | 75 | mcg |
| Molybdenum | 37.5 | mcg |
| Selenium | 10 | mcg |
| Vanadium | 5 | mcg |
| Silicon | 5 | mcg |
| Nickel | 12.5 | mcg |
| Boron | 75 | mcg |
| Copper oxide | 0.76% | |
| Copper | 1 | mg |
| Ferrous fumerate 60% | 26.96% | |
| Iron | 18 | mg |
| Dicalcium phosphate anhydrous 29.46% Ca | 61.04% | |
| Calcium | 30 | mg |
| Stearic Acid | 1.44% | |

In one embodiment shown in Example II, the core includes effective amounts of the second agents shown as a mineral trituration and excipients. In this embodiment, manganese, silicon, phosphorous, chromium, molybdenum, selenium, nickel, boron, copper and vanadium are selected as second agents.

In one embodiment, the core is formed by passing the second agents and excipients (i.e., ingredients) through a sieve and blending the ingredients until uniform. In one embodiment, the ingredients of Example II are passed through a US #4 sieve in the amounts shown and blended into a uniform mixture. In an alternative embodiment, all the ingredients of Example II except for Magnesium Stearate are passed through a US #4 sieve and blended until uniform. In an embodiment where the Magnesium Stearate is not included in the initial uniform mixture, Magnesium Stearate is passed through a US #16 sieve and added to the previously blended ingredients.

Once all the ingredients are added, the mixture is blended for a period of, for example, approximately one to two minutes. The mixture is then transferred to a suitable tablet press for single step production with the outer layer or a suitable stand alone tablet press. A suitable tablet press for the single step production may be for example a Manistry Dry Coata 900. A suitable stand alone tablet press may be for example an IPR Series press. Tooling and dies used in this example may be a one-quarter inch bisect round manufactured by for example Natoli. The dimension of the core, however, is not limited to this size. The core tablet may be made smaller or larger depending on the agents used. The tablet press may be run to a compression force of 1400 pounds at 4000 tablets per minute on a dual discharge press. The resulting hardness of the core tablet, may be, for example, seven sc with a zero percent friability. The resulting dissolution rate of a core made by the above method may be, for example, six hours. In an alternative embodiment, the core may be formulated for immediate and/or sustained release.

In an embodiment where the outer layer is applied to the core, the core is formed according to the above-described method. The outer layer includes the first agents as shown in Example I. The outer layer may be a powder form. The powder form of the outer layer may include the first agents in the amount shown in Example I. In this embodiment, the core uniform mixture described-above is transferred to a tablet press such as the Manistry Dry Coata 900 for single step production.

In an alternative embodiment, the tablet is produced in two separate processes. In this embodiment, the uniform core mixture is transferred to a Korsch XL 800 tablet press with a #C option for coating with the outer layer. In one embodiment, production is accomplished via the Korsch XL 800, a compressed core tablet is delivered via conveyer to the tablet press. The outer layer is delivered in the powder form to the press. Dies are filled with half of the amount of the outer layer mixture shown in Example I (i.e., 435.5 mg total). The outer layer mixture is then tamped to remove any excess air and flattened to prepare a surface for placement of the core. The core tablet is then delivered to the die and placed on the surface of outer layer. The remainder of the outer layer mixture is delivered to an opposite side of the core. A compression force of 1500 psi may be used to yield the hybrid tablet having an outer layer and core. In one embodiment, the final tablet has a hardness sc of approximately 25. The tablet may further have a friability of approximately 0.06 percent (%). The outer layer of the tablet may have a dissolution rate, for example, of approximately 45 seconds.

The above-described hybrid tablet is suitable for administration to a person in connection with a method of controlling the release of agents within the body and incorporating several agents within one tablet. The tablets are further suitable for swallowing in the absence of a liquid. In one embodiment, the tablet may be taken as a dietary supplement in addition to meals.

In the preceding detailed description, specific embodiments are described. It will, however, be evident that various modifications and changes may be made thereto without departing from the broader spirit and scope of the claims. The specification and drawings are, accordingly, to be regarded in an illustrative rather than restrictive sense.

What is claimed is:

1. A hybrid tablet comprising:
an outer layer comprising a first agent, the outer layer comprising a first dissolution rate; and
an inner core comprising a second agent, the inner core comprising a second dissolution rate that is different than the first dissolution rate.

2. The tablet of claim 1, wherein the first dissolution rate is greater than the second dissolution rate.

3. The tablet of claim 1, wherein the outer layer comprises a property rendering it palatably acceptable for dissolution in the buccal cavity.

4. The tablet of claim 1, wherein the inner core is one of a bilayer tablet and a single layer tablet.

5. The tablet of claim 1, wherein the first agent is selected from the group consisting of a vitamin, a therapeutic, a mineral, an herbal extract, an amino acid, a stimulant and a drug product.

6. The tablet of claim 1, wherein the second agent is selected from the group consisting of a mineral, a vitamin, a probiotic and an herbal extract.

7. The tablet of claim 1, wherein the second agent is in a liquid form.

8. The tablet of claim 1, wherein the outer layer comprises an effective amount of an organic acid suitable to elicit a salivary response within the mouth to aid in swallowing the inner core in the absence of liquids.

9. The tablet of claim 1, wherein the inner core further comprises a delay release agent.

10. The tablet of claim 4, wherein the bilayer of the inner core comprises:
a first layer having a second dissolution rate; and
a second layer having a third dissolution rate, wherein the second dissolution rate is different from the third dissolution rate.

11. The tablet of claim 1, wherein the inner core is one of a soft gelatin capsule and a gum.

12. The tablet of claim 1, wherein the outer layer further comprises beads having a fourth dissolution rate that is different than one of the first dissolution rate and the second dissolution rate.

13. The tablet of claims 5 or 6, wherein the vitamin is selected from the group consisting of ascorbic acid, CoQ10, vitamin A, vitamin B, vitamin D and vitamin K.

14. The tablet of claim 6, wherein the herbal extract is selected from the group consisting of green tea extract, cinnamon extract, Echinacea extract, ginseng extract and Andrographis extract.

15. The tablet of claim 6, wherein the mineral is selected from the group consisting of calcium carbonate and calcium citrate.

16. The tablet of claim 5, wherein the amino acid is L-arginine and the stimulant is caffeine.

17. The tablet of claim 6, wherein the probiotic is acidophilus.

18. The tablet of claim 5, wherein the therapeutic is selected from the group consisting of a fish oil and glucosamine.

19. The tablet of claim 5, wherein the mineral is selected from the group consisting of zinc gluconate, ferrous sulfate, chromium picolinate and magnesium oxide.

20. The tablet of claim 5, wherein the herbal extract is one of Yerba Santa extract and cinnamon extract.

21. The tablet of claim 11, wherein the second agent is selected from the group consisting of a vitamin, a therapeutic, lecithin, an herbal extract and a carotenoid, wherein the vitamin is selected from the group consisting of a vitamin A and a vitamin E, the therapeutic is a fish oil, the herbal extract is evening primrose oil and the carotenoid is lycopene.

22. A method comprising:
forming an outer layer having a first agent, wherein the outer layer elicits a salivary response in a mouth;
forming a core having a second agent, wherein the core is formulated for timed-release of the second agent; and
applying the outer layer to the core.

23. The method of claim 22, wherein the second agent is released over a period of approximately 24 hours or less.

24. The method of claim 22, further comprising:
selecting the second agent from the group consisting of a vitamin, a therapeutic, a mineral, an herbal extract, an amino acid, a stimulant and a drug product.

25. The method of claim 22, further comprising:
selecting the first agent from the group consisting of a mineral, a vitamin, a probiotic and an herbal extract.
